(19) United States
(12) Patent Application Publication
(10) Pub. No.: US 2020/0188276 A1
(43) Pub. Date: Jun. 18, 2020

(54) COMPOSITION AND METHOD FOR TEMPORARILY RESHAPING KERATINOUS FIBERS

(71) Applicant: Henkel AG & Co. KGaA, Duesseldorf (DE)

(72) Inventors: JULIA BIBIANE LANGE, Bad Bramstedt (DE); CYRIELLE MARTINEZ, Hamburg (DE); DIANE METTEN, Hamburg (DE)

(73) Assignee: Henkel AG & Co. KGaA, Duesseldorf (DE)

(21) Appl. No.: 16/717,675

(22) Filed: Dec. 17, 2019

(30) Foreign Application Priority Data
Dec. 18, 2018 (DE) 10 2018 222 051.4

(51) Int. Cl. A61K 8/73; A61K 8/36; A61K 8/81; A61Q 5/06; A61K 8/41

(52) U.S. Cl. CPC A61K 8/737; A61K 8/36; A61K 8/8182; A61Q 5/06; A61K 2800/5426; A61K 8/41; A61K 8/8176; A61K 2800/594; A61K 8/8135

(57) ABSTRACT

A cosmetic composition for temporarily reshaping keratin fibers, containing:
PS a) a cationically modified guar derivative with a weight-average molecular weight in the range of from about 5,000 to about 200,000 and a degree of cationic substitution in the range of from about 0.1 to about 2, and
b) at least one cationic, vinylcaprolactam-containing copolymer.

A use thereof and a method of utilizing such a composition.

### CROSS-REFERENCE TO RELATED APPLICATION

This application claims priority to German Patent Application No. 10 2018 222 051.4, filed Dec. 18, 2018, which is incorporated herein by reference in its entirety.

### TECHNICAL FIELD

The present disclosure relates to a cosmetic composition based on two selected polymers for firming hair or for temporarily reshaping keratin fibers, in particular human hair, and a method utilizing this composition.

### BACKGROUND

The temporary shaping of hairstyles for a relatively long period of time of up to several days generally requires the application of firming active ingredients. Hair treatment agents that serve to temporarily shape the hair thus play an important role. Appropriate compositions for temporary reshaping usually contain synthetic polymers and/or wax as firming active ingredients. Compositions for assisting the temporary reshaping of keratinous fibers can be provided for example in the form of hairspray, hair wax, hair gel or hair mousse.

The most important property of a composition for temporarily reshaping hair, also referred to hereinafter as styling agents, lies in providing the treated hair with the greatest possible hold in the newly modeled form—i.e. a form impressed on the hair. Reference is also made to a strong hairstyle hold or to a high holding power of the styling agent. The hold of a hairstyle is determined fundamentally by the type and quantity of the firming active ingredient used, however the further constituents of the styling agent can also have an effect.

In addition to a high holding power, styling agents must also satisfy a wide range of further requirements. These can be divided roughly into properties on the hair, properties of the particular formulation, for example properties of the mousse, of the gel, or of the sprayed aerosol, and properties that concern the handling of the styling agent, wherein the properties on the hair are attributed particular importance. In particular, moisture resistance, low stickiness (tack), and a balanced conditioning effect can be cited. Furthermore, a styling agent should be universally usable for all hair types where possible and should be mild on the hair and skin.

The hairstyle hold in general and, in the case of wavy hair, the curl retention are particular requirements of styling agents. In this context “curl retention” is a measure of the degree of hold of curls. Curl retention is usually worse if the treated hair is in humid conditions, because the tendency of hair to absorb moisture, i.e., water, causes the hair strands to hang limp

In order to satisfy the different requirements, a multiplicity of synthetic polymers which are used in styling agents have already been developed as firming active ingredients. These polymers can be divided into cationic, anionic, non-ionic and amphoteric firming polymers.

A styling agent based on a selected polymer combination, comprising a cationic vinylcaprolactam-containing copolymer is described by international patent application WO 2013/092062 A2.

### BRIEF SUMMARY

Cosmetic compositions and methods of using the same are provided. In an exemplary embodiment, a cosmetic composition includes a cationically modified guar derivative a) and a cationic, vinylcaprolactam-containing copolymer b). The cationically modified guar derivative a) has a weight-average molecular weight in the range of from about 5,000 to about 200,000 and a degree of cationic substitution in the range of from about 0.1 to about 2.

A method of using a cosmetic composition is provided in another embodiment. The method includes applying the cosmetic composition to keratinous fibers, and fixing the keratinous fibers in a shape. The cosmetic composition includes a cationically modified guar derivative a) with a weight-average molecular weight in the range of from about 5,000 to about 200,000 and a degree of cationic substitution in the range of from about 0.1 to about 2. The cosmetic composition also includes a cationic, vinylcaprolactam-containing copolymer b).

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

One object of the present disclosure was to provide a further suitable polymer combination which is exemplified by good film-forming and/or firming properties, a high degree of hold without having to compromise on flexibility, and good moisture resistance—in particular resistance to perspiration and water. In particular, one object of the present disclosure is to provide styling agents of this kind which provide both high long-term hold and high curl retention in humid conditions.

This has been achieved as contemplated herein by a combination of two selected polymers.

The following is provided by the present disclosure:

1. A cosmetic composition for temporarily reshaping keratin fibers, containing:

a) at least one cationically modified guar derivative a) with a weight-average molecular weight in the range of from about 5,000 to about 200,000 and a degree of cationic substitution in the range of from about 0.1 to about 2, and
b) at least one cationic, vinylcaprolactam-containing copolymer b).

2. The cosmetic composition as contemplated herein, wherein the weight proportion of the cationically modified guar derivative a) in the total weight of the composition is from about 0.1 to about 10% by weight, preferably from about 0.15 to about 5% by weight, and in particular from about 0.2 to about 2.5% by weight.

3. The cosmetic composition according to either one of the preceding points, wherein the cationically modified guar derivative a) has a weight-average molecular weight in the range of from about 20,000 to about 150,000, more preferably in the range of from about 35,000 to about 100,000, and very particularly preferably in the range of from about 50,000 to about 70,000.

4. The cosmetic composition as contemplated herein, wherein the cationically modified guar derivative a) has a degree of cationic substitution in the range of from about 0.2 to about 1.

5. The cosmetic composition as contemplated herein, wherein the cationically modified guar derivative a) is selected from the group of compounds with the INCI name Guar Hydroxypropyltrimonium Chloride.

6. The cosmetic composition as contemplated herein, wherein the weight proportion of the copolymer b) in the total weight of the composition is from about 0.1 to about 10% by weight, preferably from about 0.15 to about 5% by weight, and in particular from about 0.2 to about 2.5% by weight.

7. The cosmetic composition as contemplated herein, wherein the copolymer b) comprises at least one structural unit of formula (I), at least one structural unit of formula (II), at least one structural unit of formula (III) and at least one structural unit of formula (IV),

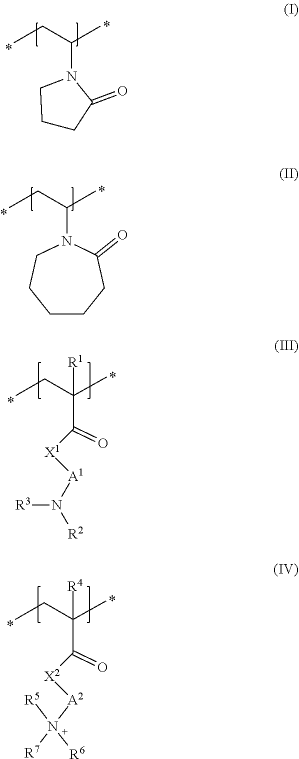

in which
R¹ and R⁴ independently of one another stand for a hydrogen atom or a methyl group,
X¹ and X² independently of one another stand for an oxygen atom or an NH group,
A¹ and A² independently of one another stand for an ethane-1,2-diyl, propane-1,3-diyl or butane-1,4-diyl group,
R², R³, R⁵ and R⁶ independently of one another stand for a (C₁ to C₄) alkyl group, and
R⁷ stands for a (C₈-C₃₀) alkyl group.

8. The cosmetic composition as contemplated herein, wherein the copolymer b) is selected from the group of compounds with the INCI name Polyquaternium-69.

9. The cosmetic composition as contemplated herein, wherein the weight ratio of cationically modified guar derivative a) to copolymer b) is from about 10:1 to about 1:10, preferably from about 5:1 to about 1:5, more preferably from about 3:1 to about 1:3, and in particular preferably from about 1.5:1 to about 1:1.5.

10. The cosmetic composition as contemplated herein, also contains

c) polyvinylpyrrolidone and/or vinylpyrrolidone/vinyl acetate copolymer, preferably polyvinylpyrrolidone.

11. The cosmetic composition according to point 10, wherein the weight proportion of the polyvinylpyrrolidone and/or vinylpyrrolidone/vinyl acetate copolymer c) in the total weight of the cosmetic composition is from about 0.1 to about 10% by weight, preferably from about 2 to about 8.5% by weight, and in particular from about 3 to about 7% by weight.

12. The cosmetic composition as contemplated herein, wherein the composition, based on its total weight, contains from about 0.01 to about 5% by weight, preferably from about 0.02 to about 4% by weight, and in particular from about 0.05 to about 2% by weight of an organic acid or salt thereof, preferably lactic acid or a salt thereof.

13. The cosmetic composition as contemplated herein, wherein the cosmetic composition, based on its total weight, contains from about 0.01 to about 5% by weight, more preferably from about 0.01 to about 2% by weight, and in particular preferably from about 0.02 to about 1.5% by weight of an alkanolamine or neutralized form thereof, in particular 2-amino-2-methylpropanol or a neutralized form thereof.

14. The cosmetic composition as contemplated herein, wherein the composition, based on its total weight, contains at least about 20% by weight, preferably at least about 40% by weight, and in particular at least about 65% by weight of water.

15. The cosmetic composition as contemplated herein, exemplified in that the composition is present in the form of a hair gel, a hairspray, a hair mousse, a hair cream or a hair wax.

16. Use of a cosmetic composition as contemplated herein for temporarily reshaping keratinous fibers, in particular human hair.

17. Use of a cosmetic composition as contemplated herein for improving the moisture resistance of temporarily reshaped keratin fibers.

18. Use of a cosmetic composition as contemplated herein for improving the high humidity curl retention of temporarily reshaped keratin fibers.

19. A method for temporarily reshaping keratinous fibers, in particular human hair, in which a cosmetic composition as contemplated herein is applied to the keratin fibers and the form of said fibers is temporarily fixed.

Cationic guar derivatives are used in haircare products to nourish the hair, for example to make the hair easier to comb. In skincare products the cationic guar derivatives can impart conditioning effects on the skin. In laundry detergent and fabric softener formulations, cationic guar derivatives impart conditioning, softening, abrasion-resistant and antistatic properties on the fabrics treated with them.

Even more surprisingly it has been found that selected cationic guar derivatives in combination with a firming/film-forming styling polymer, such as the copolymer b), which is already used in styling products, can result in a high hold of styling products. Other properties usually required of styling products, such as low tack, are retained. A good combination of properties of this kind was not anticipated, not even in the knowledge of the individual components, and was surprising. It has also been found by way of combination of the two components that an effect going significantly beyond a mere additive effect of said components, i.e. a synergistic effect, is obtained in respect of the moisture resistance, in particular in respect of the high humidity curl retention, and was demonstrated in the HHRC test (high humidity curl retention test).

The term “keratin fibers” as contemplated herein comprises fur, wool and feathers, but in particular human hair. In this context human hair may include head hair and/or beard hair.

The key constituents of the cosmetic composition are the cationic guar derivative a) and the cationic vinylcaprolactam-containing copolymer b),

Compared to alternative cosmetic compositions the cosmetic compositions are exemplified in particular by an improved high humidity curl retention. A weight ratio of the cationically modified guar derivative a) and of the copolymer b) in the cosmetic composition of from about 10:1 to about 1:10, preferably from about 5:1 to about 1:5, and in particular preferably from about 3:1 to about 1:3 has proven to be particularly advantageous for the cosmetic properties of the compositions. It is extremely advantageous if the weight ratio of the cationically modified guar derivative a) to copolymer b) is about 1:1, in particular is in the range of from about 1.1:1 to about 1:1.1.

The cosmetic compositions, as a first compulsory component, contain a cationic guar derivative a).

Within the scope of this application the term “guar derivatives” is understood to mean (bio)chemically and/or physically modified guar gum. Guar gum is a polysaccharide formed of galactose and mannose which has a linear backbone of β-1,4-linked mannose units. Galactose units are bound to each second mannose unit of this backbone by β-1,6 glycosidic bonds. The chemical modification of these guar gums can be achieved for example by esterification or etherification of the hydroxy groups of the polysaccharide or by reaction with bases, acids or oxidizing agents. A biochemical modification of these guar gums can be achieved for example by reaction with hydrolytic enzymes, bacteria or fungi. A physical modification is possible for example with use of heat, radiation and comminution, for example by employing high-speed agitators.

The term “cationically modified guar derivatives” is understood to mean guar gums of which the hydroxy groups have been esterified or etherified with a compound having at least one cationic group. This cationic group may be either permanently cationic or temporarily cationic. Compounds which comprise a cationic group irrespective of the pH value of the cosmetic composition are referred to as “permanently cationic” as contemplated herein. These include in particular compounds with quaternary nitrogen atoms, such as quaternary ammonium groups. By contrast, compounds which have a cationic group only at certain pH values, in particular pH values in the acidic range, are referred to as “temporarily cationic”. Examples of temporarily cationic groups are amino groups.

The cationic functionality can be added by a wide range of methods. For example, the starting material may be reacted for a sufficiently long time and at sufficient temperature with a tertiary amino compound or a quaternary ammonium compound that contains groups able to react with reactive groups of the guar, in particular the hydroxy groups.

Suitable compounds for introducing the cationic functionality include, for example, 2-dialkylaminoethyl chloride and quaternary ammonium compounds such as 3-chloro-2-hydroxypropyl trimethylammonium chloride and 2,3-epoxypropyl trimethylammonium chloride. Further examples are glycidyl trialkylammonium salts and 3-halogen-2-hydroxypropyl trialkylammonium salts such as glycidyl trimethylammonium chloride, glycidyl triethylammonium chloride, glycidyl tripropylammonium chloride, glycidyl ethyl dimethylammonium chloride, glycidyl diethyl methylammonium chloride and the corresponding bromides and iodides; 3-chloro-2-hydroxypropyl trimethylammonium chloride, 3-chloro-2-hydroxypropyl triethylammonium chloride, 3-chloro-2-hydroxypropyl tripropylammonium chloride, 3-chloro-2-hydroxypropyl ethyl dimethylammonium chloride and the corresponding bromides and iodides thereof; and quaternary ammonium compounds, such as halides of imidazoline ring-containing compounds.

It is preferred that the cationically modified guar derivative a) comprises at least one structural unit of formula (I),

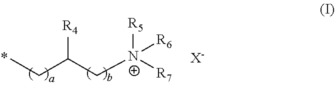

in which
R₄ stands for hydrogen, a C₁₋₄ alkyl group or a hydroxyl group,
R₅, R₆ and R₇ independently of one another stand for a C₁₋₈ alkyl group,
a and b independently of one another stand for integers from 1 to 3, and
X− stands for a physiologically acceptable anion.
Examples of C₁₋₄ alkyl groups are methyl, ethyl, propyl, isopropyl, butyl, sec-butyl, isobutyl or tert-butyl groups.
Examples of C₁₋₈ alkyl groups are methyl, ethyl, propyl, isopropyl, butyl, sec-butyl, isobutyl, tert-butyl, pentyl and hexyl, heptyl and octyl groups.

In the structural unit of formula (I) the group R₄ preferably stands for a hydroxyl group, and a and b independently of one another stand for the integer 1.

In addition, it is preferred if, in the structural unit of formula (I), the groups R₅ to R₇, independently of one another, stand for a C₁₋₆ alkyl group, preferably for a C₁₋₄ alkyl group, preferably for a C₁₋₃ alkyl group, in particular for a C₁ alkyl group, and X⁻ stands for a halide ion, in particular chloride.

Other derivatizations of the cationic guar derivative a) with non-ionic substituents, that is to say hydroxyalkyl, in which the alkyl is a straight or branched hydrocarbon group having 1 to 6 carbon atoms (for example hydroxyethyl, hydroxypropyl, hydroxybutyl), or anionic substituents, such as carboxymethyl groups, are optional. These optional substituents can be added to the cationic guar derivative a) by reaction with reagents such as (1) alkylene oxides (for example ethylene oxide, propylene oxide, butylene oxide) for obtaining hydroxyethyl groups, hydroxypropyl groups or hydroxybutyl groups or with (2) chloromethyl acetic acid to obtain a carboxymethyl group. However, it is extremely preferred if the cationic guar derivative a) does not comprise any further substituents, regardless of whether non-ionic or anionic in nature.

The cationically modified guar derivative a) has a weight-average molecular weight in the range of from about 5,000 to about 200,000. It is preferred if the cationically modified guar derivative a) has a weight-average molecular weight in the range of from about 20,000 to about 150,000, more preferably in the range of from about 35,000 to about 100,000, and very particularly preferably in the range of from about 50,000 to about 70,000.

The weight-average molecular weight can be determined for example by employing gel permeation chromatography with use of a polystyrene standard.

The cationically modified guar derivative a) has a degree of cationic substitution (DS) in the range of from about 0.1 to about 2. The degree of cationic substitution preferably lies in a range of from about 0.2 to about 1.

It is particularly advantageous if the cationically modified guar derivative a) has a degree of substitution (DS) by the structural unit of formula (I) of from about 0.1 to about 2, in particular from about 0.2 to about 1.

The degree of substitution (DS) describes the mean number of cationic structural units, in particular of cationic structural units of formula (I), which are bonded per monomer of the polysaccharide, that is to say per anhydromannose and anhydrogalactose. Since each monomer of the polysaccharide in the agent has 3 free OH groups, the DS values may assume values between 0 and 3. For example, a DS value of 1 thus means that in the agent one cationic structural unit is bonded per monomer of the polysaccharide, such that each monomer also has 2 free OH groups. The degree of substitution (DS) may be determined for example by employing ¹H-NMR spectroscopy or titration.

It is extremely preferred if the cationically modified guar derivative a) comprises a cationically modified guar derivative a) with the INCI name “Guar Hydroxypropyltrimonium Chloride”, which has a weight-average molecular weight in the range of from about 5,000 to about 200,000 and a degree of cationic substitution in the range of from about 0.1 to about 2. Such a cationically modified guar derivative a) is commercially available under the name “N-Hance CCG 45 Cationic Guar” from Ashland Specialty Chemical.

The weight proportion of the cationic guar derivative a) in the total weight of the composition is preferably from about 0.1 to about 10% by weight, particularly preferably from about 0.15 to about 5% by weight, and in particular from about 0.2 to about 2.5% by weight.

As a second essential constituent, the cosmetic compositions contain at least one cationic vinylcaprolactam-containing copolymer b).

The copolymers b) preferably comprise at least one structural unit of formula (I), at least one structural unit of formula (II), at least one structural unit of formula (III) and at least one structural unit of formula (IV),

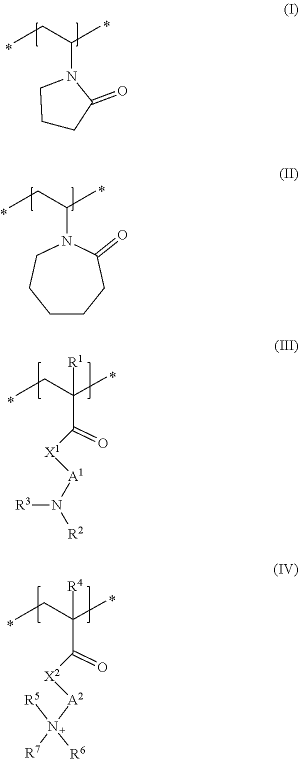

in which
R¹ and R⁴ independently of one another stand for a hydrogen atom or a methyl group,
X¹ and X² independently of one another stand for an oxygen atom or an NH group,
A¹ and A² independently of one another stand for an ethane-1,2-diyl, propane-1,3-diyl or butane-1,4-diyl group,
R², R³, R⁵ and R⁶ independently of one another stand for a (C₁ to C₄) alkyl group, and
R⁷ stands for a (C₈-C₃₀) alkyl group.

In the above formulas and all following formulas, a chemical bond which is denoted by the

symbol * stands for a free valence of the corresponding structural fragment.

In order to compensate for the positive polymer charge in the composition, all possible physiologically acceptable anions are used, such as chloride, bromide, hydrogen sulfate, methyl sulfate, ethyl sulfate, tetrafluoroborate, phosphate, hydrogen phosphate, dihydrogen phosphate or p-toluene sulfonate, or triflate.

Examples of (C₁ to C₄) alkyl groups are methyl, ethyl, propyl, isopropyl, butyl, sec-butyl, isobutyl or tert-butyl groups. Examples of (C₈ to C₃₀) alkyl groups are octyl (capryl), decyl (caprinyl), dodecyl (lauryl), tetradecyl (myristyl), hexadecyl (cetyl), octadecyl (stearyl), eicosyl (arachyl), docosyl (behenyl).

The cationic vinylcaprolactam-containing copolymers b) preferably have a molecular weight of from about 10,000 g/mol to about 5,0000,000 g/mol, in particular from about 50,000 g/mol to about 5,000,000 g/mol, particularly preferably from about 75,000 g/mol to about 1,000,000 g/mol.

It is preferred if the copolymer b) in relation to the above-mentioned formulas (I) to (IV) satisfies one or more of the following features:
R¹ and R⁴ each mean a methyl group,
X¹ stands for an HN group,
X² stands for an HN group,
A¹ and A² independently of one another stand for ethane-1,2-diyl or propane-1,3-diyl,
R², R³, R⁵ and R⁶ stand independently of one another for methyl or ethyl (particularly preferably for methyl),
R⁷ stands for a (C₁₀ to C₂₄) alkyl group, in particular for decyl (caprinyl), dodecyl (lauryl), tetradecyl (myristyl), hexadecyl (cetyl), octadecyl (stearyl), eicosyl (arachyl), or docosyl (behenyl).

It is also preferred to choose the structural unit of formula (III) from at least one structural unit of formula (III-1) to (III-8)

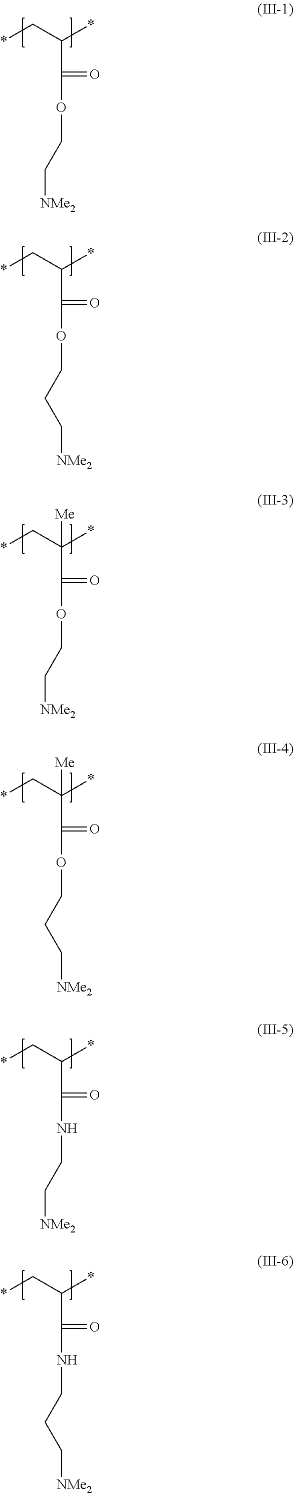

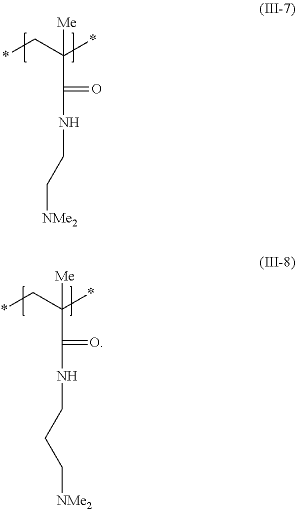

In addition, it has proven to be particularly preferred to choose the structural unit according to formula (III-7) and/or formula (III-8) as the structural unit of formula (III). The structural unit of formula (III-8) is a very particularly preferred structural unit.

In respect of the solution to the problem, it has been found to be preferable if the structural unit of formula (IV) is selected from at least one structural unit of formulas (IV-1) to (IV-8)

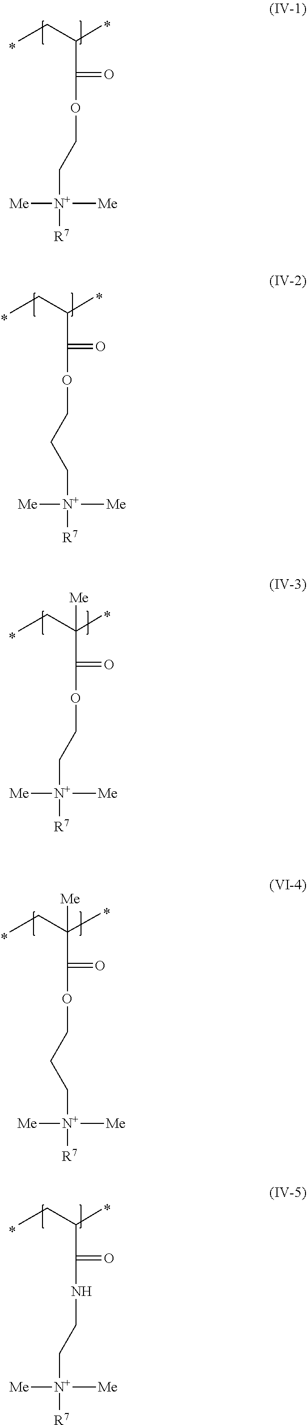

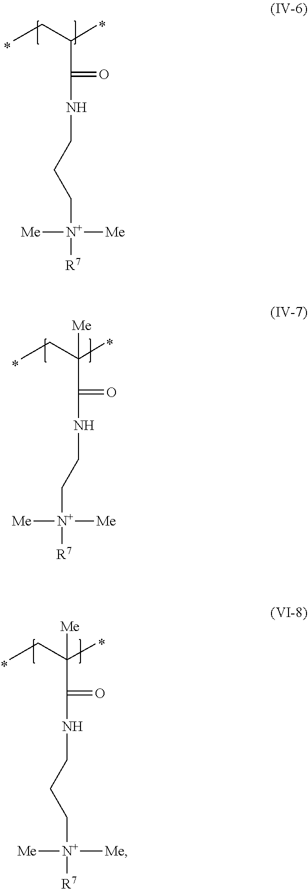

in which R⁷ stands in each case for a (C₈ to C₃₀) alkyl group.

The structural units of formula (IV-7) and/or formula (IV-8) are also particularly preferred structural units of formula (IV), in which R⁷ in each case stands for octyl (capryl), decyl (caprinyl), dodecyl (lauryl), tetradecyl (myristyl), hexadecyl (cetyl), octadecyl (stearyl), eicosyl (arachyl) or docosyl (behenyl). The structural unit of formula (IV-8) is a very particularly preferred structural unit of formula (IV).

A cationic vinylcaprolactam-containing copolymer b) used very particularly preferably in the composition comprises at least one structural unit of formula (I), at least one structural unit of formula (II), at least one structural unit of formula (III-8), and at least one structural unit of formula (IV-8),

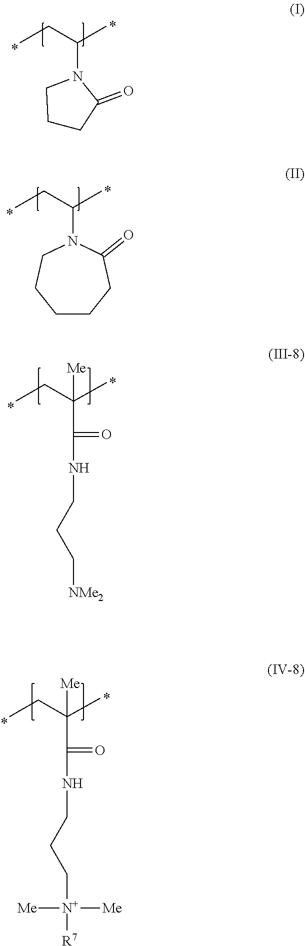

in which R⁷ stands for octyl (capryl), decyl (caprinyl), dodecyl (lauryl), tetradecyl (myristyl), hexadecyl (cetyl), octadecyl (stearyl), eicosyl (arachyl), or docosyl (behenyl).

A very particularly preferred cationic vinylcaprolactam-containing copolymer b) is the copolymer from N-vinylpyrrolidone, N-vinylcaprolactam, N-(3-dimethylaminopropyl)methacrylamide and 3-(methacryloylamino)propyl-lauryl-dimethylammonium chloride (INCI name: Polyquaternium-69), which is sold for example under the trade name AquaStyle 300 (28-32% by weight active substance in ethanol-water mixture, molecular weight 350,000) from the company Ashland.

The weight proportion of the copolymer b) in the total weight of the composition is preferably from about 0.1 to about 10% by weight, particularly preferably from about 0.15 to about 5% by weight, and in particular from about 0.2 to about 2.5% by weight.

It may be preferred if the cosmetic composition contains one or more further polymers which is/are different from the polymers a) and b) and for example support the thickening agent or the gel formation or the film formation. Examples are cationic, anionic, non-ionic or amphoteric polymers.

Examples are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polyquaternium-68, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer.

The further component acting as gel former is a homopolyacrylic acid (INCI: Carbomer), which is available for example under the name Carbopol® in different embodiments. The carbomer is preferably contained in a proportion of from about 0.05 to about 1.5% by weight, preferably from about 0.2 to about 0.8% by weight, in relation to the total weight of the cosmetic composition.

In order to further increase their cosmetic effect, besides the polymers a) and b) and an optionally added thickening agent or gel former, preferred compositions additionally contain a film-forming polymer c) different from these ingredients, in particular an anionic or non-ionic polymer c).

Examples of non-ionic polymers are:
Vinylpyrrolidone/vinyl ester copolymers, as sold for example under the name Luviskol® (BASF). Luviskol VA 64 and Luviskol VA 73, each vinylpyrrolidone/vinyl acetate copolymers, are preferred non-ionic polymers.
Cellulose ethers, such as hydroxypropyl cellulose, hydroxyethyl cellulose and methylhydroxypropyl cellulose, as are sold for example under the trade names Culminal and Benecel (AQUALON).
Shellac.
Polyvinylpyrrolidones, as are sold for example under the name Luviskol (BASF).
Siloxanes. These siloxanes can be both water-soluble and water-insoluble. Both volatile and non-volatile siloxanes are suitable, wherein compounds of which the boiling point at normal pressure is above 200° C. are understood to be non-volatile siloxanes. Preferred siloxanes are polydialkylsiloxanes, such as polydimethyl siloxane, polyalkylaryl siloxanes, such as polyphenylmethyl siloxane, ethoxylated polydialkyl siloxanes, and polydialkyl siloxanes which contain amine and/or hydroxy groups.
Glycosidically substituted silicones.

On account of their cosmetic effect in combination with the copolymers a) and b), film-forming polymers used with preference are the polyvinylpyrrolidones (INCI name: PVP) and the vinylpyrrolidone/vinyl acetate copolymers (INCI name VP/VA copolymer). Primarily the holding properties, but also the application properties of the cosmetic compositions are advantageously influenced by the addition of film-forming polymers, in particular the aforementioned polyvinylpyrrolidones and vinylpyrrolidone/vinyl acetate copolymers. The weight proportion of these polymers is preferably limited to amounts between about 1.0 and about 10% by weight. Preferred cosmetic compositions also contain, in relation to their total weight, between about 1 and about 10% by weight of polyvinylpyrrolidone and/or vinylpyrrolidone/vinyl acetate copolymer, preferably polyvinylpyrrolidone. Particularly preferred cosmetic compositions have a proportion by weight of the polyvinylpyrrolidone and/or vinylpyrrolidone/vinyl acetate copolymer c) in the total weight of the cosmetic composition of from about 2 to about 8.5% by weight, preferably from about 3 to about 7% by weight.

The cosmetic composition can contain further conventional substances of styling products. In particular, additional nourishing substances can be mentioned as further suitable auxiliaries and additives.

As a nourishing substance, the product can contain at least one protein hydrolysate and/or a derivative thereof, for example. Protein hydrolysates are product mixtures which are obtained by acid-catalyzed, base-catalyzed or enzymatically catalyzed breakdown of proteins. The term “protein hydrolysates” is understood to also mean total hydrolysates and also individual amino acids and derivatives thereof as well as mixtures of different amino acids.

As a nourishing substance, the composition as contemplated herein can also contain at least one vitamin, a provitamin, a vitamin precursor and/or one of the derivatives thereof. Here, vitamins, provitamins and vitamin precursors that are usually assigned to the groups A, B, C, E, F and H are preferred.

Similarly to the addition of glycerol and/or propylene glycol, the addition of panthenol increases the flexibility of the polymer film formed with application of the composition.

As a nourishing substance, the compositions can also contain at least one plant extract, but also monosaccharides or oligosaccharides and/or lipids.

Oil bodies are also suitable as a nourishing substance. Natural and synthetic cosmetic oil bodies include, for example, plant oils, liquid paraffin oils, isoparaffin oils and synthetic hydrocarbons as well as di-n-alkyl ethers having a total of between about 12 and about 36 C atoms, in particular from about 12 to about 24 C atoms. Preferred cosmetic compositions as contemplated herein contain at least one oil body, preferably at least one oil body from the group of silicone oils. The group of silicone oils includes, in particular, the dimethicones, which also includes the cyclomethicones, the aminofunctional silicones, and also the dimethiconols. The dimethicones can be both linear and branched and cyclic, or cyclic and branched. Suitable silicone oils or silicone gums are in particular dialkyl and alkylaryl siloxanes, such as dimethyl polysiloxane and methylphenyl polysiloxane, and the alkoxylated, quaternized, or anionic derivatives thereof. Cyclic and linear polydialkyl siloxanes, the alkoxylated and/or aminized derivatives thereof, dihydroxypolydimethyl siloxanes and polyphenyl alkyl siloxanes are preferred.

Ester oils, i.e. esters of C6-C30 fatty acids with C2-C30 fatty alcohols, preferably monoesters of the fatty acids with alcohols having 2 to about 24 C atoms, such as isopropylmyristate (Rilanit® IPM), isononanoic acid C16-18 alkyl ester (Cetiol® SN), 2-ethylhexyl palmitate (Cegesoft® 24), stearic acid-2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanite® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V) are further preferred nourishing oil bodies.

Furthermore, dicarboxylic acid esters, symmetrical, asymmetrical or cyclic esters of carbon dioxide with fatty alcohols, tri fatty acid esters of saturated and/or unsaturated linear and/or branched fatty acids with glycerol or fatty acid partial glycerides, which are understood to mean monoglycerides, diglycerides and technical mixtures thereof, are to be understood as nourishing substances.

Emulsifiers or surface-active substances are also preferably contained in the composition. PEG derivatives of hydrogenated castor oil which are obtainable for example under the name PEG Hydrogenated Castor Oil are preferred, for example PEG-30 Hydrogenated Castor Oil, PEG-33 Hydrogenated Castor Oil, PEG-35 Hydrogenated Castor Oil, PEG-36 Hydrogenated Castor Oil, or PEG-40 Hydrogenated Castor Oil. The use of PEG-40 Hydrogenated Castor Oil is preferred. These are preferably contained in an amount of from about 0.05 to about 1.5% by weight, more preferably from about 0.1 to about 1% by weight, also preferably from about 0.2 to about 0.8% by weight or from about 0.3 to about 0.6% by weight. Besides the ability to be packaged, in particular the ease with which the cosmetic compositions can be washed out is also improved by the addition of the surface-active substances, in particular the aforementioned PEG derivatives of hydrogenated castor oil.

The cosmetic compositions contain the ingredients or active ingredients in a cosmetically acceptable carrier.

Preferred cosmetically acceptable carriers are aqueous, alcoholic, or aqueous-alcoholic media with preferably at least about 10% by weight water, calculated on the basis of the total weight of the composition. The cosmetic carrier particularly preferably contains water, in particular in such an amount that the cosmetic composition, calculated on the basis of its total weight, contains at least about 20% by weight, in particular at least about 40% by weight, most preferably at least about 65% by weight of water. Cosmetic compositions that are very particularly preferred comprise, in relation to their total weight, a water content between about 50 and about 95% by weight, preferably between about 60 and about 90% by weight, and in particular between about 65 and about 85% by weight.

As alcohols, the lower alcohols with 1 to 4 carbon atoms usually used in particular for cosmetic purposes, such as ethanol and isopropanol, can be included.

Examples of water-soluble solvents as cosolvents are glycerol and/or ethylene glycol and/or 1,2-propylene glycol in an amount of from 0 to about 30% by weight in relation to the total composition.

It may be preferred if the composition contains an organic acid or salt thereof. The organic acid is preferably selected from the group of maleic acid, lactic acid, acetic acid, propanoic acid, citric acid, tartaric acid, succinic acid, oxalic acid, gluconic acid, malic acid, amino acids and mixtures thereof. The organic acid very particularly preferably comprises lactic acid.

The amount of organic acid or salt thereof, preferably of lactic acid or salt thereof, in the total weight of the cosmetic composition is preferably from about 0.01 to about 5% by weight, more preferably from about 0.02 to about 4% by weight, and in particular preferably from about 0.05 to about 2% by weight.

The cationically modified guar derivative is preferably used in the form of an acidic, aqueous solution. An organic acid is preferably used in order to acidify the aqueous solution.

It may be preferred if the cosmetic composition also contains an alkanolamine. The alkanolamines that can be used as alkalizing agents are preferably selected from primary amines having a C₂-C₆ alkyl parent substance carrying at least one hydroxyl group. Particularly preferred alkanolamines are selected from the group of 2-aminoethan-1-ol (monoethanolamine), tris(2-hydroxyethyl)-amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol. Alkanolamines that are very particularly preferred are selected from the group of 2-aminoethan-1-ol, 2-amino-2-methylpropan-1-ol and 2-amino-2-methyl-propan-1,3-diol. 2-amino-2-methylpropanol has proven to be particularly suitable. The weight proportions of the amino alcohol or its neutralized form, preferably of the 2-amino-2-methylpropanol, in the total weight of the cosmetic composition are preferably from about 0.01 to about 5% by weight, more preferably from about 0.01 to about 2% by weight, and in particular preferably from about 0.02 to about 1.5% by weight.

The composition of some preferred cosmetic compositions can be deduced from the following tables (specified amounts in % by weight are in relation to the total weight of the cosmetic composition unless specified otherwise).

| | Formula | Formula | Formula | Formula | Formula |
|---|---|---|---|---|---|
| | 1 | 2 | 3 | 4 | 5 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Misc | to 100 | to 100 | to 100 | to 100 | to 100 |

| | Formula | Formula | Formula | Formula | Formula |
|---|---|---|---|---|---|
| | 1a | 2a | 3a | 4a | 5a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| Misc | to 100 | to 100 | to 100 | to 100 | to 100 |

| | Formula | Formula | Formula | Formula | Formula |
|---|---|---|---|---|---|
| | 1b | 2b | 3b | 4b | 5b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| Misc | to 100 | to 100 | to 100 | to 100 | to 100 |

| | Formula | Formula | Formula | Formula | Formula |
|---|---|---|---|---|---|
| | 6 | 7 | 8 | 9 | 10 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| Misc | to 100 | to 100 | to 100 | to 100 | to 100 |

| | Formula | Formula | Formula | Formula | Formula |
|---|---|---|---|---|---|
| | 6a | 7a | 8a | 9a | 10a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| Misc | to 100 | to 100 | to 100 | to 100 | to 100 |

| | Formula | Formula | Formula | Formula | Formula |
|---|---|---|---|---|---|
| | 6b | 7b | 8b | 9b | 10b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| Polyvinylpyrrolidone | 1 to 10 | 2 to 8.5 | 2 to 8.5 | 3 to 7 | 3 to 7 |
| Misc | to 100 | to 100 | to 100 | to 100 | to 100 |

| | Formula | Formula | Formula | Formula | Formula |
|---|---|---|---|---|---|
| | 11 | 12 | 13 | 14 | 15 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| Misc | to 100 | to 100 | to 100 | to 100 | to 100 |

| | Formula | Formula | Formula | Formula | Formula |
|---|---|---|---|---|---|
| | 11a | 12a | 13a | 14a | 15a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| Misc | to 100 | to 100 | to 100 | to 100 | to 100 |

| | Formula | Formula | Formula | Formula | Formula |
|---|---|---|---|---|---|
| | 11b | 12b | 13b | 14b | 15b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| Vinylpyrrolidone/vinyl | 1 to 10 | 2 to 8.5 | 2 to 8.5 | 3 to 7 | 3 to 7 |
| acetate copolymer | | | | | |
| Misc | to 100 | to 100 | to 100 | to 100 | to 100 |

| | Formula | Formula | Formula | Formula | Formula |
|---|---|---|---|---|---|
| | 16 | 17 | 18 | 19 | 20 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Misc | to 100 | to 100 | to 100 | to 100 | to 100 |

| | Formula | Formula | Formula | Formula | Formula |
|---|---|---|---|---|---|
| | 16a | 17a | 18a | 19a | 20a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Misc | to 100 | to 100 | to 100 | to 100 | to 100 |

| | Formula | Formula | Formula | Formula | Formula |
|---|---|---|---|---|---|
| | 16b | 17b | 18b | 19b | 20b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| Carbomer | 0.02 to 3 | 0.05 to 2 | 0.05 to 1.5 | 0.2 to 1.5 | 0.2 to 0.8 |
| Misc | to 100 | to 100 | to 100 | to 100 | to 100 |

| | Formula | Formula | Formula | Formula | Formula |
|---|---|---|---|---|---|
| | 16c | 17c | 18c | 19c | 20c |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Misc | to 100 | to 100 | to 100 | to 100 | to 100 |

| | Formula | Formula | Formula | Formula | Formula |
|---|---|---|---|---|---|
| | 16d | 17d | 18d | 19d | 20d |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| Carbomer | 0.02 to 3 | 0.05 to 2 | 0.05 to 1.5 | 0.2 to 1.5 | 0.2 to 0.8 |
| Misc | to 100 | to 100 | to 100 | to 100 | to 100 |

| | Formula | Formula | Formula | Formula | Formula |
|---|---|---|---|---|---|
| | 21 | 22 | 23 | 24 | 25 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Misc | to 100 | to 100 | to 100 | to 100 | to 100 |

| | Formula | Formula | Formula | Formula | Formula |
|---|---|---|---|---|---|
| | 21a | 22a | 23 a | 24a | 25a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Misc | to 100 | to 100 | to 100 | to 100 | to 100 |

| | Formula | Formula | Formula | Formula | Formula |
|---|---|---|---|---|---|
| | 21b | 22b | 23b | 24b | 25b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Misc | to 100 | to 100 | to 100 | to 100 | to 100 |

| | Formula | Formula | Formula | Formula | Formula |
|---|---|---|---|---|---|
| | 26 | 27 | 28 | 29 | 30 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 26a | 27a | 28a | 29a | 30a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 26b | 27b | 28b | 29b | 30b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 31 | 32 | 33 | 34 | 35 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 31a | 32a | 33a | 34a | 35a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 31b | 32b | 33b | 34b | 35b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| Polyvinylpyrrolidone | 1 to 10 | 2 to 8.5 | 2 to 8.5 | 3 to 7 | 3 to 7 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 36 | 37 | 38 | 39 | 40 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 36a | 37a | 38a | 39a | 40a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 36b | 37b | 38b | 39b | 40b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| Vinylpyrrolidone/vinyl | 1 to 10 | 2 to 8.5 | 2 to 8.5 | 3 to 7 | 3 to 7 |
| acetate copolymer | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 41 | 42 | 43 | 44 | 45 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 41a | 42a | 43a | 44a | 45a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 41b | 42b | 43b | 44b | 45b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 46 | 47 | 48 | 49 | 50 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 46a | 47a | 48a | 49a | 50a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 46b | 47b | 48b | 49b | 50b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 51 | 52 | 53 | 54 | 55 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 51a | 52a | 53a | 54a | 55a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 51b | 52b | 53b | 54b | 55b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| Polyvinylpyrrolidone | 1 to 10 | 2 to 8.5 | 2 to 8.5 | 3 to 7 | 3 to 7 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 56 | 57 | 58 | 59 | 60 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 56a | 57a | 58a | 59a | 60a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyquaternium-69 | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 56b | 57b | 58b | 59b | 60b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| AquaStyle 300 | 0.1 to 10 | 0.15 to 5 | 0.15 to 5 | 0.2 to 2.5 | 0.2 to 2.5 |
| (specified as solid | | | | | |
| content) | | | | | |
| Vinylpyrrolidone/vinyl | 1 to 10 | 2 to 8.5 | 2 to 8.5 | 3 to 7 | 3 to 7 |
| acetate copolymer | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |

* cationically modified guar derivative with a weight-average molecular weight in the range of from about 5,000 to about 200,000 and a degree of cationic substitution in the range of from about 0.1 to about 2
** with a weight-average molecular weight in the range of from about 5,000 to about 200,000 and a degree of cationic substitution (DS) in the range of from about 0.1 to about 2

The entry “Misc” is to be understood to mean a cosmetic carrier, in particular water (unless listed separately) and optionally further usual constituents of styling products.

The cosmetic composition of the present disclosure can be provided in the forms that are usual for the temporary reshaping of hair, for example as a hair gel, hairspray, hair mousse, hair cream or hair wax. The cosmetic composition is preferably provided as a hair gel.

Both hair mousses and hairsprays require the presence of propellants. However, preferably no hydrocarbons or only small quantities of hydrocarbons should be used for this. Propane, propane/butane mixtures and dimethyl ethers are particularly suitable propellants.

The present disclosure also relates to the use of cosmetic compositions as contemplated herein for temporarily reshaping keratin fibers, in particular human hair, and to a method for temporarily reshaping keratin fibers, in particular human hair, in which the cosmetic composition as contemplated herein is applied to the keratin fibers and the form of said fibers is temporarily fixed.

With regard to further preferred embodiments of the use and the method, that which has been said in relation to the cosmetic compositions applies mutatis mutandis.

A further subject of this patent application is the use of a cosmetic composition as contemplated herein to improve the high humidity curl retention (HHCR) of temporarily reshaped keratin fibers.

### Examples

I. The following hair gels were prepared:

| Component/raw | | | | |
|---|---|---|---|---|
| material | INCI name or chemical name | V1 | V2 | E1 |
| N-Hance CCG 45 ¹ | Guar Hydroxypropyltrimonium | 1 | — | 0.5 |
| | Chloride | | | |
| AquaStyle 300 ² | Polyquaternium-69 | — | 3.34 | 1.67 |
| Water | | 99 | 96.66 | 97.83 |
| Total | | 100 | 100 | 100 |

¹ 92% by weight active substance in water
² 28-32% by weight active substance in water

The amounts specified in the table are given in % by weight of the respective raw materials, in relation to the total composition.

The curl retention in humid conditions of cleaned strands of Kerling hair was determined for the obtained styling agents by employing an HHCR test (high humidity curl retention test: 6 h; mean value from 5 hair strands in each case):

| | V1 | V2 | E1 |
|---|---|---|---|
| HHCR | 19.4% | 50.3% | 55.2% |

The polymer combination E1 therefore demonstrated a synergistic effect, going considerably beyond a purely additive effect, in respect of high humidity curl retention.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for temporarily reshaping keratin fibers comprising:
a) at least one cationically modified guar derivative a) with a weight-average molecular weight in the range of from about 5,000 to about 200,000 and a degree of cationic substitution in the range of from about 0.1 to about 2; and
b) at least one cationic, vinylcaprolactam-containing copolymer b).

2. The cosmetic composition according to claim 1, wherein a weight proportion of the at least one cationically modified guar derivative a) in a total weight of the composition is from about 0.1 to about 10% by weight.

3. The cosmetic composition according to claim 1, wherein the at least one cationically modified guar derivative a) is selected from the group of compounds with the INCI name Guar Hydroxypropyltrimonium Chloride.

4. The cosmetic composition according to claim 1, wherein a weight proportion of the at least one copolymer b) in a total weight of the composition is from about 0.1 to about 10% by weight.

5. The cosmetic composition according to claim 1, wherein the at least one copolymer b) is selected from the group of compounds with the INCI name Polyquaternium-69.

6. The cosmetic composition according to claim 1, wherein the at least one copolymer b) comprises at least one structural unit of formula (I), at least one structural unit of formula (II), at least one structural unit of formula (III), and at least one structural unit of formula (IV), 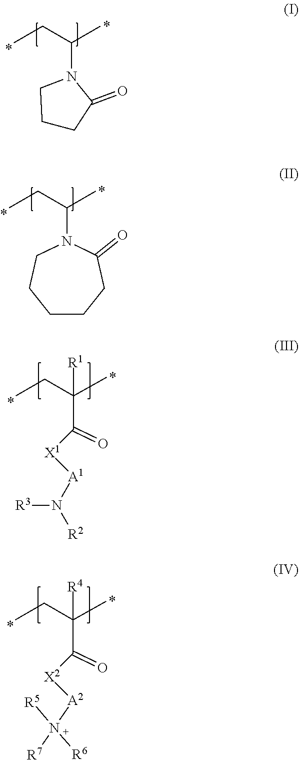
in which
R¹ and R⁴ independently of one another stand for a hydrogen atom or a methyl group,
X¹ and X² independently of one another stand for an oxygen atom or an NH group,
A1 and A2 independently of one another stand for an ethane-1,2-diyl, propane-1,3-diyl or butane-1,4-diyl group,
R², R³, R⁵ and R⁶ independently of one another stand for a (C₁ to C₄) alkyl group, and
R⁷ stands for a (C₈-C₃₀) alkyl group.

7. The cosmetic composition according to claim 1, wherein the cosmetic composition also comprises an alkanolamine or neutralized form thereof.

8. The cosmetic composition according to claim 1, wherein the composition also comprises an organic acid or salt thereof.

9. A method of using a cosmetic composition, the method comprising the steps of:
applying the cosmetic composition to keratinous fibers, wherein the cosmetic composition comprises a cationically modified guar derivative a) with a weight-average molecular weight in the range of from about 5,000 to about 200,000 and a degree of cationic substitution in the range of from about 0.1 to about 2, and a cationic, vinylcaprolactam-containing copolymer b); and
fixing the keratinous fibers in a shape.

10. The method of claim 9, wherein: applying the cosmetic composition comprises applying the cosmetic composition wherein the cosmetic composition comprises the cationically modified guar derivative a) at from about 0.2 to about 2.5 wt. %, and wherein the cosmetic composition comprises the copolymer b) at from about 0.2 to about 2.5 wt. %, where the weight percent's are based on a total weight of the cosmetic composition.

11. The method of claim 10, wherein: applying the cosmetic composition comprises applying the cosmetic composition wherein the cationically modified guar derivative a) is a compound with the INCI name Guar Hydroxypropyltrimonium Chloride, and wherein the copolymer b) is a compound with the INCI name Polyquaternium-69.

12. The cosmetic composition of claim 1, wherein the at least one cationically modified guar derivative a) is present in the cosmetic composition at from about 0.2 to about 2.5 wt. %, based on a total weight of the cosmetic composition.

13. The cosmetic composition of claim 1, wherein the at least one copolymer b) is present in the cosmetic composition at from about 0.2 to about 2.5 wt. %, based on a total weight of the cosmetic composition.

14. The cosmetic composition of claim 1, further comprising a polymer c), where the polymer c) is selected from the group of polyvinylpyrrolidone, vinylpyrrolidone/vinyl acetate copolymer, and combinations thereof.

15. The cosmetic composition of claim 14, wherein the polymer c) is present in the cosmetic composition at from about 0.1 to about 10 wt. %, based on a total weight of the cosmetic composition.

16. The cosmetic composition of claim 14, wherein the polymer c) is polyvinylpyrrolidone.

17. The cosmetic composition of claim 1, wherein the at least one cationically modified guar derivative a) is present in the cosmetic composition at from about 0.15 to about 5 wt. %, based on a total weight of the cosmetic composition.

18. The cosmetic composition of claim 1, wherein the at least one copolymer b) is present in the cosmetic composition at from about 0.15 to about 5 wt. %, based on a total weight of the cosmetic composition.

19. The cosmetic composition of claim 1, wherein the at least one cationically modified guar derivative a) is a compound with the INCI name Guar Hydroxypropyltrimonium Chloride, wherein the at least one cationically modified guar derivative a) is present in the cosmetic composition at from about 0.2 to about 2.5 wt. %, based on a total weight of the cosmetic composition, wherein the at least one copolymer b) is a compound with the INCI name Polyquaternium-69, and wherein the at least one copolymer b) is present in the cosmetic composition at from about 0.2 to about 2.5 wt. %, based on the total weight of the cosmetic composition.

20. The cosmetic composition of claim 1, wherein a weight ratio of the at least one cationically modified guar derivative a) to the at least one copolymer b) is from about 3:1 to about 1:3.
